(19) United States
(12) Patent Application Publication
(10) Pub. No.: US 2015/0283374 A1
(43) Pub. Date: Oct. 8, 2015

(54) Electric Feedthrough For Electromedical Implants, Electric Contact Element Comprising Such A Feedthrough, And Electromedical Implant

(71) Applicant: BIOTRONIK SE & Co. KG, Berlin (DE)

(72) Inventors: Daniel Kronmueller, Nuernberg (DE); Josef Teske, Hallstadt (DE)

(21) Appl. No.: 14/632,369

(22) Filed: Feb. 26, 2015

Related U.S. Application Data
(60) Provisional application No. 61/973,878, filed on Apr. 2, 2014.

(51) Int. Cl. A61N 1/05; H02G 15/013

(52) U.S. Cl. CPC A61N 1/05; H02G 15/013

(57) ABSTRACT

An electric feedthrough for electromedical implants, including at least one electric contact for connection to a mating contact. The at least one electric contact is formed as a conductive track which extends at least in regions in or on a dielectric substrate from a first region to a second region, wherein the substrate, when transitioning from the first into the second region, is guided through a flange, and in that the substrate is connected to the in a hermetically sealed manner. Also provided is an electric contact element and an electromedical implant including such a feedthrough.

### CROSS-REFERENCE TO RELATED APPLICATIONS

This patent application claims the benefit of co-pending U.S. Provisional Patent Application No. 61/973,878, filed on Apr. 2, 2014, which is hereby incorporated by reference in its entirety.

### TECHNICAL FIELD

The present invention relates to an electric feedthrough for electromedical implants, an electric contact element comprising such a feedthrough, and an electromedical implant comprising a contact element of this type.

### BACKGROUND

A wide range of medical implants are known from the prior art. In conjunction with the present invention, an electromedical implant is understood to be an implant which, besides a power supply (for example, a battery), also comprises further electrical and/or electronic components, which are arranged in a housing that is hermetically sealed.

Since any intervention involves stress for the patient, increased demands are being placed on implants in terms of durability and reliability. Only materials that are biocompatible, that is to say that cannot be absorbed or metabolized by the human organism, may be used on the outer face of the implant. Known biocompatible materials include, but are not limited to, titanium or platinum, for example.

Electromedical implants of this type include, for example, cardiac pacemakers, defibrillators, neurostimulators, cardiac pacemakers, cardioverter/defibrillators and cochlear implants.

After implantation in a patient, the implants treat the patient by monitoring or actively stimulating the electronic pulses of the body. For example, stimulation pulses or defibrillator shocks are thus transmitted or delivered to specific points of the body. Further, the electronic potentials of points of the body can be detected and recorded and, after electronic evaluation, can be made available to the treating doctor via an antenna on a monitoring system, for example, what is known as a home monitoring system. Electric connections from the outer face of the implant to the hermetically sealed inner region, in which the signal processing takes place, are necessary for all of these purposes.

Feedthroughs for electromedical implants in which metal pins are passed through a ceramic body for electric signal guidance are known from the prior art, for example, documents European Patent No. EP 1 897 588 B1 and U.S. Publication No. 2013/0309237. These pins have to be inserted and soldered in a complex manner. The metal solders form menisci and, therefore, the pins cannot be packed very densely due to the necessary insulation distances. Furthermore, the inserted pins are not stable in terms of their position relative to one another and relative to the ceramic or the housing, since the pins deform slightly. This impairs the attachment by machine and in an automated manner and causes additional costs. Further, it is disadvantageous that circuit board and ceramic feedthroughs have to be produced in separate manufacturing steps and joined in a complex manner, and that the electric connections have to be linked. With the use of EMI filters with such a feedthrough, complex joining processes (e.g., soft soldering, adhesive bonding, welding), which result in costs and errors, have to be used with such a feedthrough in order to attach EMI filters to feedthroughs. Inter alia, add-on components (e.g., capacitors) are also necessary on the connecting strips and are elaborate in terms of the space required.

The same is also true for glass feedthroughs or glass-ceramic feedthroughs, which are likewise known, wherein the control of the glass solder flow is additionally difficult with some material combinations of the glass feedthroughs. With both feedthrough types, limitations in respect of the feasible geometry and the selection of materials are necessary due to the generally high soldering temperature and the involved coefficients of thermal expansion.

The present invention is directed toward overcoming one or more of the above-mentioned problems.

### SUMMARY

One object of the present invention is to create a high-quality and robust electric feedthrough for electromedical implants that can be produced in a reproducible manner.

A further object of the present invention is to provide an electric contact element comprising such a feedthrough.

A further object is to provide an electromedical implant comprising such a contact element.

At least one of these objects is achieved in accordance with the present invention by the features of the independent claim(s). Favorable embodiments and advantages of the present invention will emerge from the further claims and the description.

An electric feedthrough for electromedical implants is proposed, comprising at least one electric contact for connection to a mating contact, the at least one electric contact being formed as a conductive track, which extends at least in regions in or on a dielectric substrate from a first region to a second region, wherein the substrate, when transitioning from the first into the second region, is guided through a flange, and wherein the substrate is connected to the flange in a hermetically sealed manner.

In particular, the seal separates the two regions from one another in a hermetically sealed manner.

The production of such a feedthrough is more cost effective than conventional multi-pin variants, in which the electric contacts are formed by individual solid wire pins. In contrast thereto, the present invention comprises electric contacts which are fixed in their position as conductive tracks on the rigid substrate and, for example, cannot deform or be deformed with respect to one another. The substrate may be single-layered, or two or more layers may be arranged on one another, wherein each layer may comprise conductive tracks. Through-platings between various substrate layers are advantageously possible.

During operation, these feedthroughs are less susceptible to faults than multi-pin variants. The possible contact density is much greater than with conventional feedthroughs. A data rate can be achieved that may be much higher with the feedthrough according to the present invention than with wire pins. The relative alignment of all electric contacts with one another is ensured. Impedance properties of the feedthrough can be adjusted selectively and can be improved. An electromagnetic shielding can also be better ensured. The electric properties, such as resistance, shielding and impedance, can be planned and optimized with the aid of the substrate, even during the design process.

The high demands on medical implants can be met due to the hermetic seal of the connection between the substrate and flange. In particular, the feedthrough is virus-proof, impermeable to water vapor, and gas-tight. Such a hermetically sealed feedthrough also meets the demands for vacuum-tight feedthroughs, in particular, for ultra-high vacuum-tight feedthroughs.

In particular, the substrate may be a printed, single-layer or multi-layer circuit board. The use of engineering ceramics as substrate material is particularly favorable. Here, what are known as LTCC ceramics (LTCC=low-temperature co-fired ceramic) or HTCC ceramics (HTCC=high-temperature co-fired ceramic) can be used. Conductive tracks and/or circuits in a number of planes formed of ceramic substrate layers can be joined together in the stage of a green compact together with a metallization, and can then be sintered jointly. Various engineering ceramics (for example, glass ceramic, AlN, Al₂O₃, etc.) are available as base material and can be structured first. They are then coated or printed with materials having a high melting point (for example, Au, Pd, Pt, Ir, Nb, Ta, W, Mo or alloys consisting of two or more components therefrom) before they are stacked to form two or more layers and are sintered. Typical thicknesses of individual layers are between 100 μm to 200 μm, in particular, between 100 μm and 150 μm.

A feedthrough comprising a plurality of electric contact elements is advantageously achieved, in which the contact elements, in contrast to conventional wire pins, do not deform and do not have to be straightened. Additional costs produced by the fitting of a plug on a circuit board can be avoided. These costs are produced, for example, because a new insulation ceramic has to be used and, in addition, pins or prongs have to be guided through the ceramic in order to produce a defined electric connection. Many individual technical processes are necessary for such a known feedthrough. Advantageously, the feedthrough according to the present invention further allows the arrangement of the conductive tracks and of the electric contacts in the first region to deviate from that of the conductive tracks in the second region.

The known finished feedthrough is additionally prone to deformation of the pins, which necessitates costly re-working measures. The electric properties of the pins or prongs relative to one another are dependent on the position of the pins and can vary easily and in an undesired manner. A transition from the circuit board to the feedthrough changes the electric properties and, likewise, the shielding, and may be the cause of EMC problems, since the unshielded pins act as antennas. The feedthrough according to the present invention advantageously avoids or reduces problems of this type.

The substrate serves as a carrier for circuit arrangements and positions the conductive tracks, which replace the pins, relative to and absolutely in the plug. Further, the substrate may serve as a resilient element which, due to an inclined position, generates a slight contact pressure on the mating plug. The electric properties in the substrate material can be set with high accuracy and in a reproducible manner and are not subject to any significant change as a result of aging or wear. EMC problems can be reduced due to the manufacture of the feedthrough with the substrate material. High data rates or transmission rates are made possible due to appropriate arrangement of the contacts and conductive tracks.

A much higher packing density of the feedthrough is possible. Costly processes during production, such as the alignment of pins, pin bending, etc., can be spared. The feedthrough can be better shielded with respect to EMC interferences.

In accordance with a favorable embodiment, a solder, in particular, a glass solder or metal solder, can be provided for the hermetically sealed connection. To this end, biocompatible materials can be used advantageously, for example, gold solder, biocompatible glass, or an alloy of TiCuNi.

In accordance with a favorable embodiment, the substrate may be a printed circuit board, in particular, a ceramic printed circuit board, and further in particular, a printed circuit board made of LTCC or HTCC ceramic. Circuit arrangements and component arrangements as are known and have been proven in electronics production can be used advantageously.

In accordance with a favorable embodiment, the substrate may comprise one or more printed circuits. Different functions can thus integrated advantageously into the feedthrough, for example, high-frequency filter, capacitors and the like. In accordance with a favorable embodiment, the substrate may also comprise one or more SMD components, which are mounted on a substrate surface. Favorable circuit arrangements formed of integrated and/discreet electric components can be provided advantageously.

In accordance with a favorable embodiment, at least the first region of the substrate may comprise an electromagnetic shielding. This can be provided by a complete metal or metalized bordering of the first region, similarly to that with a USB2.0 or HDMI plug, or a shielding can be provided within the substrate. It may also be that only the second region comprises a shielding.

In accordance with a favorable embodiment, the substrate may comprise one or more electric contacts on one or more surfaces. Electric contacts on the main faces, but also on side faces or end faces, may thus be provided.

In accordance with a further favorable embodiment, the substrate, in the first and/or second region, may have a stepped surface comprising at least two steps. This advantageously allows the possible electric contact possibilities to be extended. An unambiguous mounting position of the feedthrough is also achieved. This is advantageous, in particular, if conductive tracks and/or circuits are provided within the substrate and it is therefore necessary to distinguish the first region from the second region of the substrate.

In accordance with a favorable embodiment, the substrate may have at least one recess in the first and/or second region, such that two or more substrate segments distanced from one another are formed. This variant also allows the possible electric contact possibilities to be extended and allows an unambiguous unmistakable mounting position and attachment of the feedthrough.

In accordance with a favorable embodiment, the substrate and/or the electric contacts may be formed at least in regions of biocompatible material or may be encapsulated at least in regions by biocompatible material. The substrate may advantageously be formed from aluminum oxide Al₂O₃, silicon dioxide SiO₂, aluminum nitride AlN, an LTCC, HTCC ceramic or a glass ceramic, or may be coated thereby at least in regions.

In accordance with a favorable embodiment, the electric contacts in the two regions can be allocated differently or in a swapped manner since the conductive tracks in the substrate are electrically insulated in various conductive track planes by means of through-platings and are formed in a crossed manner. This allows a flexible design of the contacting of the device.

In accordance with a favorable embodiment, the metal of the electric conductive track may be a metal from the group of gold, platinum, iridium, palladium, niobium, tantalum, tungsten, titanium, copper, nickel or an alloy with at least one of these metals. These metals belong to the group of biocompatible materials. For example, a TiCuNi alloy can be used.

In accordance with a favorable embodiment, the flange may be metallically conductive and may preferably consist of a metal which corresponds largely to the metal of a housing of a therapy device for which the feedthrough is intended. With a metallically conductive flange, a hermetically sealed connection to the substrate can be produced, for example, by hard soldering, soft soldering or welding. If the flange is formed from the housing material, an integrally bonded and therefore hermetically sealed connection between the feedthrough and housing can be produced particularly easily and reliably.

In accordance with a favorable embodiment, the substrate may comprise an electric filter component. This enables improved interference suppression of attachments, for example, to antennas, wherein the signals can be guided completely on a closed circuit board. Active or passive filter circuit arrangements can also be placed immediately in the vicinity of the feedthrough. A filter component is advantageous, for example, for what are known as home monitoring systems, in which the implant transmits data from the site of implantation, for example, to a doctor.

In accordance with a further aspect of the present invention, an electric contact element for an electromedical implant comprising an electric feedthrough according to the present invention is proposed, comprising a substrate and at least one flange which is connected to the substrate in a hermetically sealed manner. The electric connection is advantageously plugged and can be opened or closed quickly. For a permanent connection, a plugged connection can be fixed in a defined manner, for example, by welding, soldering, in particular, hard soldering or soft soldering, deformation or the like.

Here, the substrate may be a circuit board, in particular, a printed circuit board (PCB). This can be formed such that a narrow branch is guided through the flange as a first region of the substrate and a region of greater area, as a second region, comprises components and/or circuit arrangements, which perform the functions of the electromedical implant. It is advantageous if circuit boards are used which contain exclusively biocompatible materials, for example, with Al₂O₃ as carrier material and niobium for conductive tracks.

In accordance with a favorable embodiment, the feedthrough can be coupled to a separate circuit board. The substrate and the feedthrough can therefore be structured relatively simply. More complex functions can be integrated on the separate circuit board. Contact element interchangeable parts can thus be produced in large numbers in a cost-saving manner for a wide range of different circuit boards and/or implants.

In accordance with a further aspect of the present invention, an electromedical implant, in particular, an implantable electrotherapy device is proposed, in particular, a cardiac pacemaker or cardioverter/defibrillator, comprising an electric contact element according to the present invention.

In addition, electromedical implants such as, for example, defibrillators, neurostimulators, cardiac pacemakers and cochlear implants comprising one or more electric contact elements according to the present invention are also advantageous.

Further features, aspects, objects, advantages, and possible applications of the present invention will become apparent from a study of the exemplary embodiments and examples described below, in combination with the Figures, and the appended claims.

### DESCRIPTION OF THE DRAWINGS

The present invention will be explained in greater detail hereinafter by way of example on the basis of exemplary embodiments illustrated in drawings, in which:

FIG. 1 shows a schematic plan view of a cross section through an exemplary embodiment of an electromedical implant comprising an electric contact element with a feedthrough comprising conductive tracks on a substrate as electric contact elements.

FIG. 2 shows a schematic view from an end face of the exemplary embodiment from FIG. 1.

FIG. 3 shows a schematic exploded view of a feedthrough before insertion into a flange.

FIG. 4 schematically shows variants of possible contact elements and circuit arrangements, combined, in a substrate of a feedthrough, in a single feedthrough with steps and recesses for unambiguous installation.

FIG. 5 schematically shows a section through a substrate with conductive tracks shielded within the substrate.

FIG. 6 schematically shows a section through a substrate with shielded conductive tracks within the substrate and through-platings between various layers of the substrate.

FIG. 7 schematically shows a plan view of a formed substrate with flange and shielding of electric contact elements in the form of conductive tracks on a second region of the substrate.

FIG. 8 schematically shows a variant of a first region of a feedthrough.

FIG. 9 schematically shows a further variant of a first region of a feedthrough for fastening to a circuit board.

FIG. 10 schematically shows a further variant of a first region of a feedthrough for fastening to a circuit board.

FIG. 11 schematically shows a section through a flange with fed-through substrate, which is connected to the flange in a hermetically sealed manner by means of a metal solder, comprising a conductive track on the surface of the substrate and cover layer above the conductive track.

FIG. 12 schematically shows a section through a flange with fed-through substrate, which is connected to the flange in a hermetically sealed manner by means of a metal solder, comprising a ground line on the surface of the substrate.

FIG. 13 schematically shows a section through a flange with fed-through substrate, which is connected to the flange in a hermetically sealed manner by means of a glass solder.

FIG. 14 schematically shows a double connection of a substrate to a flange.

FIG. 15 schematically shows a plan view of signal lines on a substrate, which are surrounded by a ground line.

FIG. 16 schematically shows a simplified equivalent circuit diagram of the arrangement in FIG. 15.

FIG. 17 schematically shows a section through a multi-layered substrate comprising a number of planes of ground lines.

### DETAILED DESCRIPTION

In the figures, functionally like or similarly acting elements are denoted in each case by like reference signs. The figures are schematic illustrations of the invention. They do not show specific parameters of the invention. Furthermore, the figures merely reproduce typical embodiments of the present invention and are not intended to limit the present invention to the embodiments illustrated.

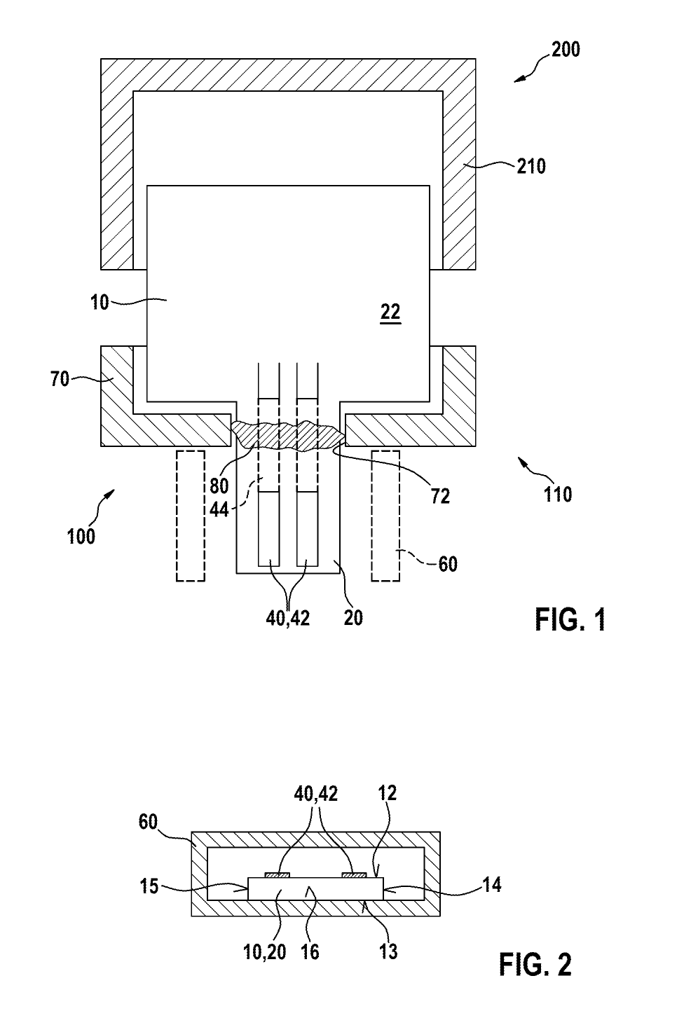

FIG. 1, as a plan view, shows a cross section through an exemplary embodiment of an electromedical implant 200 comprising an electric contact element 110 with a feedthrough 100 comprising two conductive tracks 42 on a substrate 10 as electric contact elements 40.

The conductive tracks 42 extend at least in regions in, or on, an electrically insulating substrate 10 from a first region 20 to a second region 22. The first region 20 forms an external contact, to which a mating contact (not illustrated) can be connected. For example, the first region 20 of the substrate 10 constitutes the contact of a plug, wherein the conductive tracks 42 serve as electric contact elements. The conductive tracks 42 are formed, for example, of a platinum/iridium alloy or another biocompatible material. The conductive tracks 42 are connected fixedly to the substrate 10 and do not protrude freely beyond the substrate 10, irrespective of their thicknesses perpendicular to the substrate and, therefore, the absolute and relative position of the conductive tracks 42 relative to one another is predetermined in a fixed manner in this way.

The substrate 10, as it transitions from the first region 20 into the second region 22, is guided in this exemplary embodiment through a flange 70. The flange 70 is in particular a metal flange, preferably made of titanium or another biocompatible metal material, of which the composition preferably largely matches the material of the implant housing 210.

The substrate 10 is connected to the flange 70 in a hermetically sealed manner by introducing a solder 80 into the gap between the substrate 10 and flange opening 72. The substrate 10 can be joined or soldered, for example, to the flange.

The conductive tracks 42, in the region of the passage through the flange 70, run beneath the substrate surface as buried conductive tracks 44, such that there is no electric contact between the flange 70 and the conductive tracks 42 if a metal solder 80 is inserted. Alternatively, the conductive tracks 42 may be covered by an insulation layer, and/or an electrically insulating glass solder can be used, wherein, in this case, the conductive tracks 42 do not have to be formed as a buried embodiment 44.

The first region 20 of the substrate 10 is shielded in the shown example by surrounding the upper face 12 (to be seen in plan view in FIG. 1) and lower face 13 and the two end faces 14, 15 of the substrate 10 by a metal shielding 60. FIG. 2 shows a view from the end face 16 of the substrate 10 in the first region 20. The shielding 60 on the one hand protects the first region 20 of the substrate 10 and on the other hand serves to provide an optionally fixed connection to a mating plug.

Possibilities for producing the substrate 10 include, but is not limited to, milling a contour of a circuit board formed from Al₂O₃, as is known from the field of power electronics, or already providing the green compact with this contour. Instead of such an HTCC ceramic, an LTCC ceramic may alternatively be provided, wherein, in this case, the green compact is composed from a mixture of ceramic powder, glass frit and organic binder.

An HTCC ceramic is usually then suitable if materials having a higher melting point, such as, for example, Mo, W, Ta, Nb or alloys thereof, are used as metals for the conductive tracks 42. Conventional sintering temperatures in this case lie in the range from 1,400° C. to 1,500° C. LTCC ceramics can then be considered if a metal having a relatively low melting point is used for the conductive tracks 42 or contacts 40, etc., for example, Au, Pt, Ir, Pd or alloys thereof, such that the sintering temperatures must lie below the melting point of the metal. Typical process temperatures then lie around 900° C. Ti is preferable as flange material. The solder 80, by means of which the flange 70 is connected to the substrate 10 in a hermetically sealed manner, has a lower melting point or melting range than the flange 70 if it is metal solder. If the solder 80 consists of glass, the processing temperature of the glass then lies below the melting point of the flange 70.

If materials that are not biocompatible are used in the external region 20 for the conductive tracks 42 or the contacts 40, the conductive tracks 42 or the contacts 40 are coated with a biocompatible metal or a biocompatible metal alloy, such that the non-biocompatible regions of the conductive tracks 42 or of the contacts 40 are encased completely and without gaps by biocompatible material, such as, for example, Au, Pt, Pd, Ir, Ti, Nb, Ta or alloys thereof. Possible methods for applying such a coating are dependent on the selection of the basic material of the conductive tracks 42, said coating being applied, for example, by means of galvanization or by means of a masking process in a PVD method, that is to say by means of vapor deposition or cathode sputtering. Alternatively, circuit board material of which the conductive tracks 42 already consist of biocompatible material can be used. The circuit board produced may optionally also be fitted and soldered with electric components in a reflow and SMT method. The circuit board is then inserted into a biocompatible flange 70. The flange can be produced by turning, milling, MiM technology (metal-insulator-metal technology), sintering or other methods. With a suitable solder 80 (glass solder or, for example, gold solder), the circuit board 10 is fused or joined into the flange 70. Here, the dimensional accuracy and the stress distribution of the metal pairing is to be considered expediently in order to form permanently hermitically sealed feedthroughs 100. The feedthrough 100 can then be connected to the rest of the housing 210 in a hermetically sealed and mechanically fixed manner, for example, by means of hard soldering, soft soldering, or welding, in particular, laser welding.

For example, a feedthrough 100 with the electronics of what is known as a leadless cardiac pacemaker can thus be produced and joined directly into the housing 210, whereby costs can be saved and flexible, pluggable electrodes can be spared.

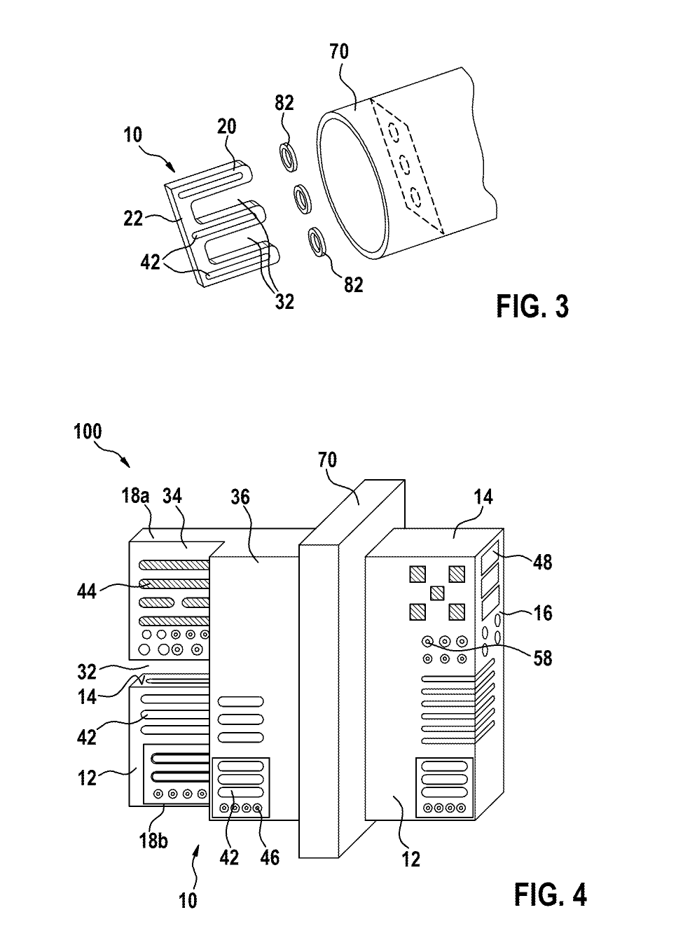

FIG. 3, as an exploded illustration, shows a variant of a feedthrough 100 with the substrate 10 before insertion into a flange 70, corresponding to a plug-through variant. The flange 70 can be joined in a hermetically sealed and mechanically fixed manner into an opening of an implant.

In this example, the first region 20 of the substrate 10 is formed such that recesses 32 are provided in the substrate 10 between the conductive tracks 42. The recesses 32 can be punched or milled from the green compact. The first region 20 thus has a comb-like structure. The second region 22 may have a straight edge without recesses. Solder rings 82 are fitted over the individual comb teeth and are connected to the flange 70 in a hermetically sealed and mechanically fixed manner by means of a high-temperature soldering process. The conductive tracks 42 in the first region 20 form electric contacts 40 for an electric connection to a corresponding mating element.

The substrate 10 may be a passive carrier for the conductive tracks 42 or may contain one or more circuits. The conductive tracks 42, as described with respect to FIG. 1, may be manufactured and, in the case of non-biocompatible conductive tracks, may comprise biocompatible coatings which are deposited galvanically or by means of conventional PVD or CVD layer deposition methods, such as, for example, vapor deposition, cathode sputtering or plasma-assisted methods.

FIG. 4 shows variants of possible contacts 40, conductive tracks 42 and circuit arrangements, which are combined in a single feedthrough 100 by way of example in a substrate 10 of a feedthrough 100. The substrate 10 is provided in a region with steps 34, 36, such that the substrate 10 is thinner at the free end than at the flange 70. Further, a recess 32 which divides the substrate 10 into two segments 18a, 18b is arranged there. Both of the steps 34, 36 and the recess 32 can each be used for unambiguous installation of the substrate 10 or the feedthrough 100.

Conductive tracks 42 can be provided on the upper face 12 (and/or the lower face) of the substrate 10, and/or also on side walls 14. Buried conductive tracks 14 may run within the substrate 10. Ground lines 46 can be arranged around conductive tracks 42. Further, through-platings 58 (vias) can be provided, that is to say openings into which solder can be introduced in order to interconnect conductive tracks on two different substrate layers. Furthermore, openings may lead to cavities in the substrate which can be filled with metal solder, whereby electric and biocompatible properties of the conductive tracks and contacts of the substrate 10 can be selectively adjusted.

Conductive tracks can be guided over an edge, as is illustrated in the right half of the image. Contact pads 48 can also be arranged on the end face 16 of the substrate.

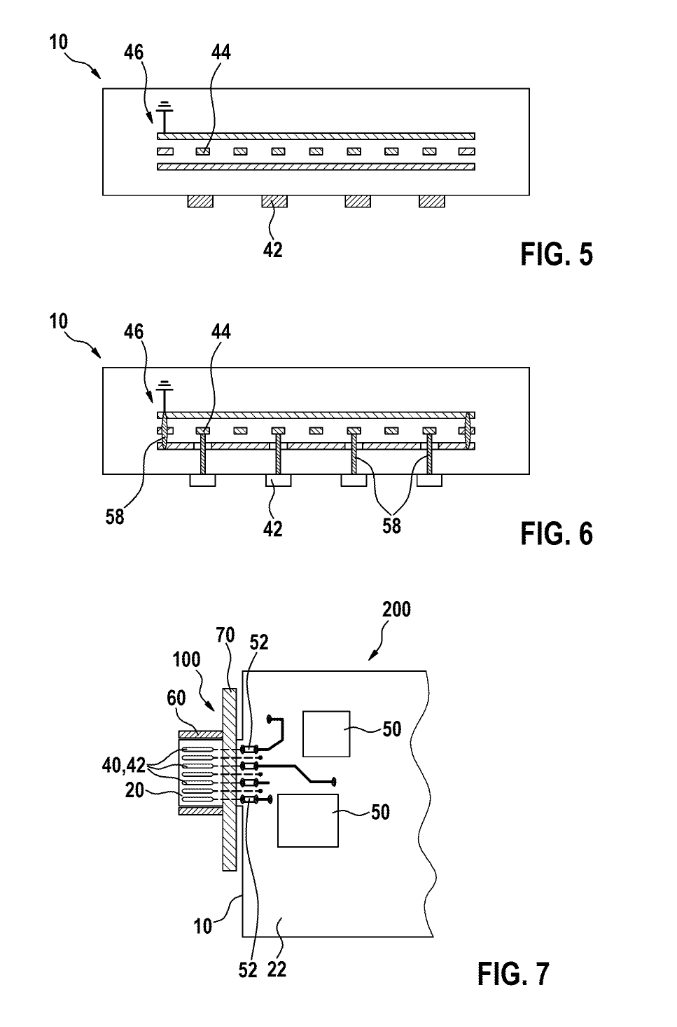

FIGS. 5-6 illustrate possibilities in sectional illustrations of how a shielding can be implemented in the interior of the substrate 10.

FIG. 5 shows a section through the substrate 10, in which conductive tracks 44 run between two flat potential lines 46, wherein the two outer conductive tracks are assigned to the potential lines. The flat potential lines 46 can also be formed as a grid (not illustrated), such that mechanical stresses in the structure can be reduced on the one hand, and hereby the function of what is known as a “Faraday cage” also remains ensured on the other hand. One of the potential lines 46 is connected to ground, for example. The other potential line can be connected to ground or may have a different potential. Conductive tracks 42 are guided on a surface of the substrate 10 over the shielded arrangement. FIG. 6 shows a shielded arrangement of conductive tracks 44 within the substrate 10, in which through-platings 58 between the two flat potential lines 46 interconnect said potential lines and also the two lateral potential lines, such that the arrangement of buried conductive tracks 44 is enclosed by a ground cage. The conductive tracks 42 on the surface of the substrate 10 are connected via through-platings 58 to the conductive tracks 44 in the interior of the ground cage. Corresponding openings are provided in one of the flat ground lines 46 for this purpose.

FIG. 7, as a plan view, schematically shows an electromedical implant 200 (illustrated without housing) with a substrate 10 with shaped outer contour, which comprises a first region 20 on one side of a flange 70 and a second region 22 on the other side of the flange 70. The first region 20 protrudes as a tongue from the second region 22 and is surrounded by a shielding 60, which is open at the free end. A plurality of conductive tracks 42 form electric contacts 40 for a mating contact device, for example, a plug or a socket. The conductive tracks 42 are guided through the flange 70 in an electrically insulated manner from the first region 20 to the second region 22, for example, by being covered with an insulating layer (not denoted in greater detail) or by being buried in the region of the flange in an inner layer of the substrate 10. The outlet of the contacts 42 may be thicker than the conductive tracks 42, such that, when plugged with a mating plug, the contacts are not abraded, but reliable electrical contacting is ensured. The second region 22 is formed as a flat circuit board and, for example, may have a microstructured circuit arrangement. SMD components are mounted thereon at the surface. For example, the conductive tracks 42 are attached in the vicinity of the flange to capacitors 52 on the circuit board in order to produce efficient shielding against electromagnetic interfering radiation. Two microchips 50 are provided by way of example.

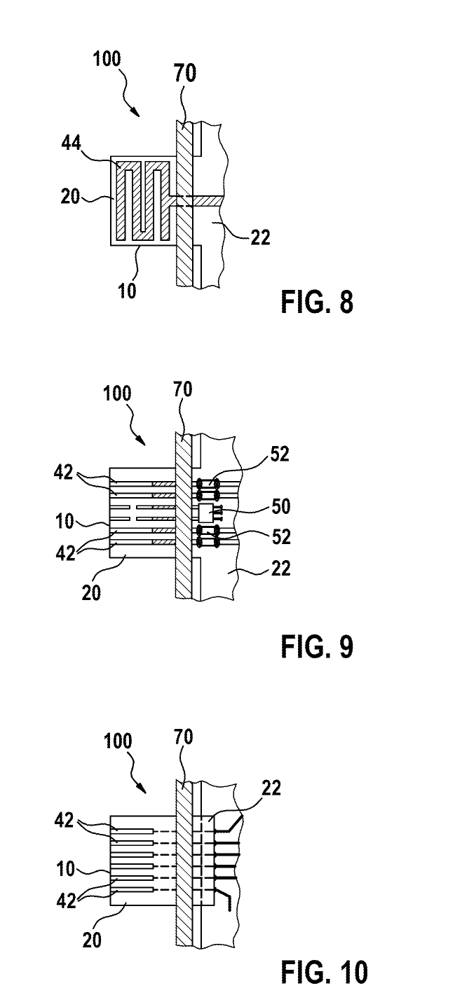

FIGS. 8-10 show various alternative feedthroughs 100 for an electromedical implant 200 from FIG. 7. FIG. 8 shows a buried antenna in the form of a meandering conductive track 44 in the first region 20 of the substrate 10. The contour of the substrate 10 is shaped as in FIG. 7, and the antenna is guided within the substrate 10 to the second region 22. The antenna can be formed as a rod or loop antenna depending on the electric demand.

In the variant in FIG. 9 the feedthrough 100 is provided for fastening on a separate circuit board (not illustrated). The substrate 10 is rectangular and, on its second region 22 in the vicinity of the flange, comprises SMD components in the form of capacitors 52 and an IC component 50. The two outer conductive tracks 4 with the capacitors in the vicinity of the flange are each guided within the circuit board 10 through the flange 70, wherein, away from the capacitors 52, connections are formed, at which bonding wires are arranged, by means of which an electric connection to the circuit board (not illustrated) can be produced. For example, a low-pass filter can be produced by means of the capacitors 52. A conductive track structure divided into two is arranged between the outer conductive tracks 42 and is assigned the IC component 50 in the second region, away from which a plurality of conductive tracks are guided to connections. The connections of the second region 22 serve for connection to the separate circuit board (not illustrated).

The variant in FIG. 10, similarly to FIG. 9, shows a feedthrough 100 which is provided for fastening on a separate circuit board (not illustrated). In the first region 20 of the substrate 10, a number of conductive tracks 42 are provided as electric contacts 40 and are not arranged on the substrate 10 equidistantly as in FIGS. 7-9, but with decreasing distance in the transverse direction. In the second region 22, connections, at which bonding wires for contacting a circuit board (not illustrated) are provided, are provided on the substrate 10 on the end face thereof. The connection of the conductive tracks 42 in the first region to the end-face connections runs within the substrate 10, such that there is no possibility of an electric short circuit between the flange 70 and the conductive tracks 42.

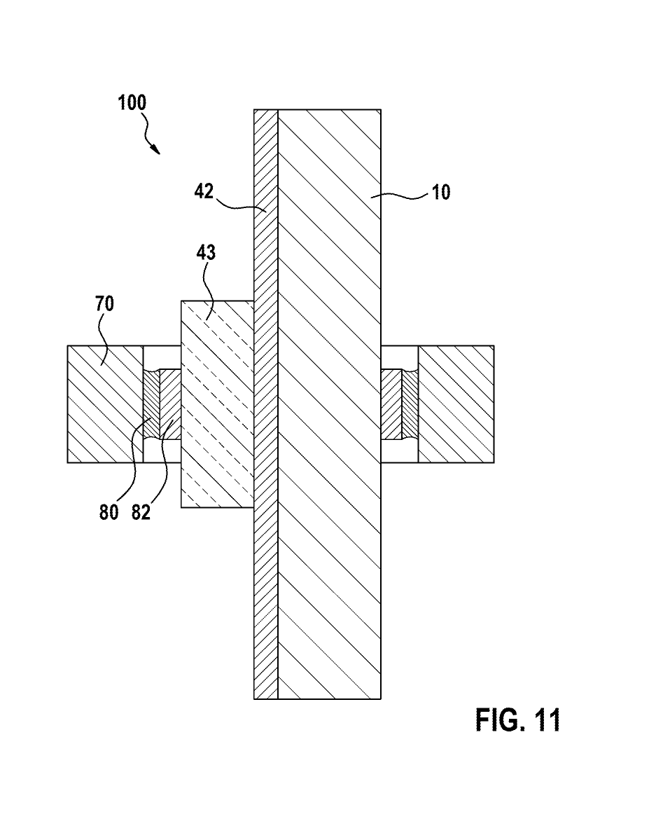

FIG. 11 shows a section through a feedthrough 100 comprising a flange 70 with fed-through substrate 10 in the manner of the exemplary embodiments of FIGS. 7-10. The flange 70 is connected to the substrate 10 in a hermetically sealed manner by means of a metal solder 80, for example, gold solder or a TiCuNi alloy. A conductive track 42 is guided on the upper face of the substrate 10 by way of example. In the region of the flange 70, the conductive track 42 is covered by an electrically insulating cover layer 43, or the substrate 10 is formed in steps, such that the conductive track 42 is buried in this region. For improved wetting of the substrate 10 and of the cover layer 43 by the solder 80, such as, for example, gold, a suitable adhesion-promoting layer 82 is provided beneath the solder 80 on the substrate 10 and on the cover layer 43. In the case of the electrically conductive metal alloy TiCuNi as solder 80, the adhesion-promoting layer 82 can be omitted, since this solder is able to wet the ceramic substrate 10 directly. The material for the adhesion-promoting layer can be selected suitably depending on the material pairing provided and, for example, consists of Nb, Ti, Ti/Mo, Ta, etc.

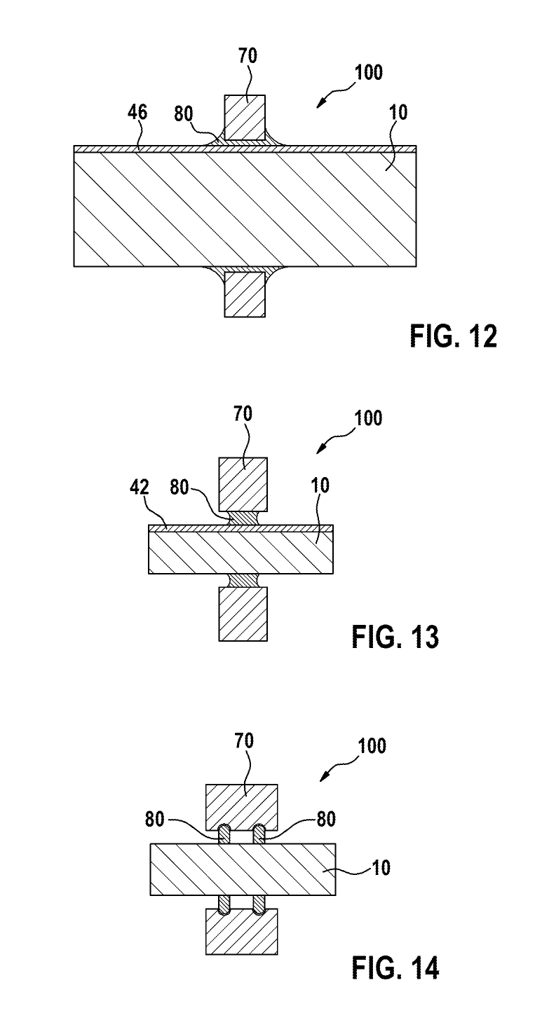

FIG. 12 shows a section through a feedthrough 100 comprising a flange 70 with substrate 10 guided through the flange 70 in the manner of the exemplary embodiments in FIGS. 7-10. The flange 70 is connected to the substrate 10 in a hermetically sealed manner by means of a metal solder 80. A ground line 46 is arranged on the surface of the substrate 10, such that the solder 80 can be connected directly to the ground line 46 and the substrate 10.

FIG. 13 shows an embodiment alternative to FIG. 12, in which, instead of a metal solder 80, a glass solder is used in order to connect the substrate 10 to the flange 70 in a hermetically sealed manner. The conductive track 42 on the substrate surface can therefore be contacted directly by the glass solder without producing an electric short circuit to the flange 70.

FIG. 14 shows a variant of a feedthrough in which the flange 70 comprises two seals, which are arranged axially in succession in the direction of the flange 70 and which are connected to the substrate 10 in a hermetically sealed manner. Each seal is produced by a soldered connection with a solder 80. An increased reliability of the hermetic connection between the flange 70 and the substrate 10 can be achieved with this variant. If one of the two hermetically sealed connections is not tight, for example, due to a crack, the crack is then limited to one seal because it cannot continue to the second seal, and the second seal remains intact, such that the hermeticity is ensured more reliably than with a single seal.

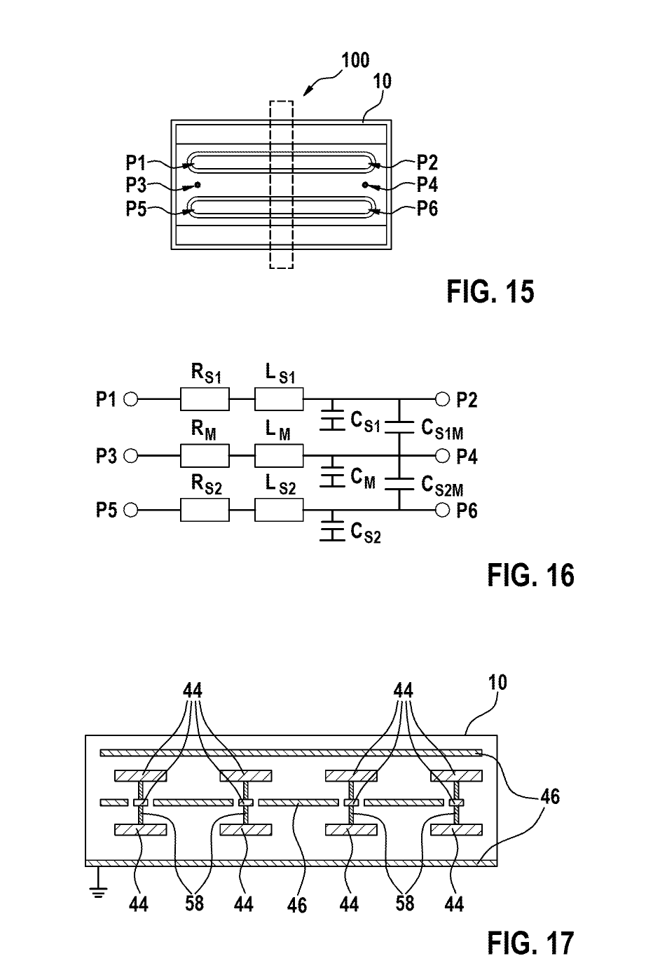

FIGS. 15-16, as a plan view (FIG. 15) and as a simplified equivalent circuit diagram (FIG. 16), show two signal lines with end points P1, P2 and P5, P6, which are arranged on a substrate 10 and are surrounded by a ground line with contact points P3, P4.

The signal line between the end points P1, P2 is composed in the equivalent circuit diagram of a resistor R_{S1}, an inductor L_{S1}, a capacitor C_{S1}, which is dependent on the substrate 10, and a capacitor C_{S1M}. The signal line between the end points P5, P6 is composed in the replacement circuit diagram of a resistor R_{S2}, an inductor L_{S2}, a capacitor C_{S2}, which is dependent on the substrate 10, and a capacitor C_{S2M}. The ground line with the contact points P3, P4 is composed in the equivalent circuit diagram of a resistor R_{M}, an inductor L_{M}, and a capacitor C_{M}.

In principle, all known methods and technologies can be used to adjust favorable and planned parameters, for example, etching, coating, printing, through-plating and the like.

FIG. 17 shows the section through a multi-layered substrate 10 with a plurality of ground planes 46 with ground lines or ground areas. The area of the conductive tracks 44 in the interior of the substrate 10 has been enlarged by additional layers and through-platings 58. For example, three layers with conductive tracks 44 are provided. The electric properties, such as damping and shielding, can be selectively varied due to the alignment and position thereof relative to the ground planes 46. The respective areas of ground 46 and conductive tracks 44 can be applied as solid areas, meanders or coating.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

### LIST OF REFERENCE NUMERALS

10 substrate
12, 13 surface
14, 15 surface
16 surface
18a, b segment
20 region
22 region
30 circuit board
32 recess
34 step
36 step
40 electric contact
42 conductive track
43 insulating layer
44 buried conductive track
46 ground
48 contact field
50 microchip
52 capacitor
54 shielding
56 filter
58 through-plating
60 shielding
62 printed circuit
70 flange
72 opening
80 solder
82 layer
100 feedthrough
110 contact element
200 electric implant
210 implant housing

I/we claim:

1. An electric feedthrough for electromedical implants, comprising:
at least one electric contact for connection to a mating contact, wherein the at least one electric contact is formed as a conductive track which extends at least in regions in or on an electrically insulating substrate from a first region to a second region, wherein the substrate, when transitioning from the first into the second region, is guided through a flange, and wherein the substrate is connected to the flange in a hermetically sealed manner.

2. The feedthrough as claimed in claim 1, wherein a solder comprising a glass solder or metal solder is provided for the hermetically sealed connection.

3. The feedthrough as claimed in claim 1, wherein the substrate is a printed circuit board comprising a ceramic printed circuit board.

4. The feedthrough as claimed in claim 1, wherein the substrate comprises one or more printed circuit arrangements.

5. The feedthrough as claimed in claim 1, wherein the substrate comprises one or more SMD components.

6. The feedthrough as claimed in claim 1, wherein at least the first region of the substrate comprises an electromagnetic shielding.

7. The feedthrough as claimed in claim 1, wherein the substrate comprises one or more electric contacts on one or more surfaces.

8. The feedthrough as claimed in claim 1, wherein the substrate, in the first and/or second region, comprises a stepped surface comprising at least two steps.

9. The feedthrough as claimed in claim 1, wherein the substrate, in the first and/or second region, comprises at least one recess, such that two or more substrate segments distanced from one another are formed.

10. The feedthrough as claimed in claim 1, wherein the substrate and/or the electric contacts is/are formed at least in regions from biocompatible material or is/are encapsulated at least in regions by biocompatible material.

11. The feedthrough as claimed in claim 1, wherein the electric contacts in the regions are allocated differently or in a swapped manner, and wherein the conductive tracks in the substrate are electrically insulated in various conductive track planes by means of through-platings and are formed in a crossed manner.

12. The feedthrough as claimed in one claim 1, wherein the metal of the electric conductive track is a metal from the group of gold, platinum, iridium, palladium, niobium, tantalum, tungsten, titanium, copper, nickel or an alloy comprising at least one of these metals.

13. The feedthrough as claimed in claim 1, wherein the flange is metallically conductive and comprises a metal which corresponds largely to the metal of a housing of a therapy device for which the feedthrough is intended.

14. The feedthrough as claimed in claim 1, wherein the substrate comprises at least one electric filter component.

15. An electric contact element for an electromedical implant comprising:
an electric feedthrough as claimed in claim lone of the preceding claims, comprising a substrate and at least one flange, which is connected to the substrate in a hermetically sealed manner.

16. The contact element as claimed in claim 15, wherein the feedthrough is coupled to a circuit board.

17. An electromedical implant including a cardiac pacemaker or cardioverter/defibrillator, comprising:
an electric contact element as claimed in claim 15.
